(19) United States
(12) Patent Application Publication
(10) Pub. No.: US 2022/0265890 A1
(43) Pub. Date: Aug. 25, 2022

(54) FLUID PROCESSING

(71) Applicant: BEADLIGHT LIMITED, Worcestershire (GB)

(72) Inventors: Christopher WILSON, Oxford (GB); Mathew CHINN, Plymouth (GB)

(21) Appl. No.: 17/732,745

(22) Filed: Apr. 29, 2022

Related U.S. Application Data
(63) Continuation of application No. PCT/GB2020/052713, filed on Oct. 27, 2020.

(30) Foreign Application Priority Data
Oct. 30, 2019 (GB) 1915782.5
Oct. 31, 2019 (GB) 1915875.7

(51) Int. Cl. A61L 9/20

(52) U.S. Cl. CPC A61L 9/20; A61L 2209/12; A61L 2209/11

(57) ABSTRACT

A method and apparatus are disclosed for at least partially purifying a fluid via a germicidal irradiation. In various aspects, the apparatus comprises at least one fluid-moving element, located in a housing, for moving a fluid through at least one fluid inlet in the housing towards at least one fluid outlet in the housing, wherein the housing comprises a dividing wall that includes at least one fluid-flow aperture in a fluid-flow pathway between the inlet and the outlet, at least one fluid-flow expansion region, and a plurality of chamber regions; at least one radiation source that provides ultraviolet radiation for irradiating the fluid in at least one of the chamber regions; and at least one full height fluid-flow blocking element, each located between two adjacent chamber regions, for at least partially blocking a fluid-flow of fluid flowing between the two adjacent chamber regions and producing a major region of turbulent fluid-flow in at least one of the two adjacent chamber regions.

The present invention relates to a method and apparatus for at least partially purifying a fluid. In particular, but not exclusively, the present invention relates to germicidal irradiation of fluid utilising an ultraviolet (UV) radiation source.

It is known that from time to time it is helpful to process a fluid to remove undesired components of that fluid. For example, a fluid could be air or oxygen that might include airborne pathogens and microorganisms. Human or general animal life forms breathing such contaminated fluid can suffer ill health consequences. As a consequence, there is a desire to be able to inactivate undesired components.

Various techniques have conventionally been suggested for processing a fluid to remove undesired components. One particular type of processing methodology utilises physical filters which include mesh-like components that trap components such as pathogens and/or microorganisms as they are caused to flow through the filter. Other techniques are known.

Many conventional techniques require filters to be replaced from time to time which can be a costly and time-consuming process. Also it is not always clear when a filtering mechanism is in need of changing. This can lead to inadequate purification during a time period when a filter mechanism is no longer functioning until it is changed.

Other conventional techniques include providing power to a filtering mechanism but these can have high power consumption requirements and it is often complex to operate such mechanisms.

Certain environments place constraints upon the manner and mechanism that can be utilised for at least partially purifying a fluid. For example, in a vehicle such as a car or aircraft a space available for purification is limited. Likewise, access to a vehicle to carry out a maintenance or service cycle can be limited.

An aircraft cabin is an example of an environment where airborne pathogens and microorganisms can cause problems to passengers due to the recycled nature of air in the confined vehicular volume. An average adult, when resting, inhales and exhales about 7 or 8 litres of air per minute. This totals around 11000 litres of air per day. Inhaled air is about 20% oxygen. Exhaled air is about 15% oxygen. Therefore about 5% of breathed air is consumed in each breath. That air is converted to carbon dioxide. Therefore human beings take in around 550 litres of pure oxygen per day. 8 litres per minute equates to 0.133 litres per second or 133 cm³/s. Air filtration systems in aircraft and aerospace environments typically rely on HEPA based filtration in order to remove airborne particles, pathogens and microorganisms from outside the aircraft as well as from air recirculated from the whole of the inside of the aircraft. It is important when any purification mechanism is utilised in such environments that the purification process does not in itself create further risks or hazards to human beings. For example, flammable by-products must be kept to a minimum and ozone production must be kept to a minimum. At times recirculated clean air will then become unclean by the time it reaches the personal airspace, due to passing throughout the whole cabin.

It is an aim of the present invention to at least partly mitigate one or more of the above-mentioned problems.

It is an aim of certain embodiments of the present invention to provide a method and apparatus for at least partially purifying a fluid.

It is an aim of certain embodiments of the present invention to provide an apparatus for at least partially purifying air or oxygen via germicidal irradiation of the fluid whereby fluid-flow in a purification device is slowed to maximise a period of time in which purification of a flow of air is carried out yet which does not unduly restrict a constant working/recycling ability for the device.

It is an aim of certain embodiments of the present invention to provide a method for purifying a fluid such as air or oxygen or the like via germicidal irradiation which can be carried out safely and conveniently and without significant power requirements or financial cost and which leaves a “cleansed” fluid without any active airborne pathogens or microorganisms.

It is an aim of certain embodiments of the present invention to provide an air filtration device which is convenient to provide in a vehicle and which has low maintenance requirements and which is discrete so as not to alarm a user of the vehicle or a passenger in the vehicle.

According to a first aspect of the present invention there is provided apparatus for at least partially purifying a fluid via germicidal irradiation of the fluid, comprising:
at least one fluid-moving element, located in a housing, for moving a fluid through at least one fluid inlet in the housing towards at least one fluid outlet in the housing, wherein the housing comprises a dividing wall that includes at least one fluid-flow aperture in a fluid-flow pathway between the inlet and the outlet, at least one fluid-flow expansion region, and a plurality of chamber regions;
at least one radiation source that provides ultraviolet radiation for irradiating the fluid in at least one of the chamber regions; and
at least one full height fluid-flow blocking element, each located between two adjacent chamber regions, for at least partially blocking a fluid-flow of fluid flowing between the two adjacent chamber regions and producing a major region of turbulent fluid-flow in at least one of the two adjacent chamber regions.

Aptly the apparatus further comprises at least one shallow fluid-flow blocking element, each located within a respective one of the chamber regions, for at least partially blocking a fluid-flow of fluid flowing within a respective chamber region and producing a minor region of turbulent fluid-flow within a respective chamber region.

Aptly the first fluid-moving element is located proximate to the fluid inlet or the fluid outlet and a further fluid-moving element is located proximate to a remainder of the fluid inlet or the fluid outlet for moving the fluid through the fluid outlet.

Aptly at least one radiation source comprises at least one radiation emitting element that emits ultraviolet radiation.

Aptly a light guide is disposed proximate to at least one radiation emitting element for diffusing emitted ultraviolet radiation.

Aptly at least one radiation source comprises a radiation filter that filters out and effectively removes ultraviolet radiation of a wavelength below 260 nanometres from the provided ultraviolet radiation.

Aptly the apparatus further comprises a controller unit, comprising a controller interface, connected to the at least one fluid-moving element and the at least one radiation providing device, for providing respective control signals to each fluid-moving element and radiation providing device.

Aptly the apparatus further comprises a power unit, comprising a power connection interface, connected to the at least one fluid-moving element and the at least one radiation providing device, for providing respective power signals to each fluid-moving element and radiation providing device.

Aptly the controller unit comprises the power unit.

Aptly the controller unit and the power unit can be one system.

Aptly the respective power signals comprise the respective control signals.

Aptly the respective power signals and the respective control signals can be transmitted across one interface.

Aptly the respective power signals and the respective control signals and be in one interface.

Aptly the controller unit can incorporate a Controlled Area Network (CAN) bus or other controller bus interface.

Aptly the housing comprises a base member and an upstanding sidewall member extending around a perimeter of the base member, and a cover member that covers the housing.

Aptly each fluid inlet and each fluid outlet comprises a through hole in the cover member.

Aptly the apparatus further comprises the fluid inlet and/or the fluid outlet comprises a slit or a row of discrete holes in the cover member and optionally the row of discrete holes or the slit is arcuate.

Aptly the apparatus further comprises each full height fluid-flow blocking element comprises a wall member upstanding from the base member or extending from the cover member and that extends between 75% and 100% of a depth of a space between an inner surface of the base member and an inner surface of the cover member.

Aptly the apparatus further comprises each full height fluid-flow blocking element has a length that is between 90% and 50% of a width between the side wall on opposed sides of the housing.

Aptly the apparatus further comprises each shallow fluid-flow blocking element comprises a bar member that extends across a whole or a portion of a width of the housing between a side wall on opposed sides of the housing and each bar member is proximate to a base member or to a cover member of the housing and has a depth between 2% and 30% of a depth of a space between an inner surface of a base member of the housing and an inner surface of a cover member of the housing.

Aptly the apparatus further comprises each shallow fluid-flow blocking element comprises one of a plurality of projections that extend from an inner surface of a cover member of the housing and/or of a base member of the housing.

Aptly each projection is a boss-like or cone-like or pin-like or dome-like element.

Aptly the apparatus includes at least one full height fluid-flow blocking element and/or at least one shallow fluid-flow blocking element and each blocking element comprises at least one vortex shedding site.

Aptly the housing comprises an antechamber between the fluid inlet and a first of the chamber regions and the housing includes mutually inclined walls that face an interior of the antechamber proximate to the fluid-flow aperture thereby providing a narrowing of the antechamber proximate to the fluid-flow aperture wherein the narrowing antechamber is narrower proximate to the fluid-flow aperture than distal to the fluid-flow aperture.

Aptly the housing comprises an exit chamber between a final chamber region of the chamber regions and the fluid outlet and the housing includes mutually inclined walls that face an interior of the exit chamber proximate to an exit aperture in a dividing wall between the final chamber region and the exit chamber thereby providing a broadening out of the exit chamber proximate to the exit aperture, the broadening out of the exit chamber being narrower proximate to the exit aperture and extending in a flared out arrangement towards a central region of the exit chamber.

According to a second aspect of the present invention there is provided a vehicle that includes apparatus for at least partially purifying a fluid via germicidal irradiation of the fluid.

Aptly the vehicle comprises an aircraft or a car or a truck or a train.

According to a third aspect of the present invention there is provided a method for at least partially purifying a fluid via germicidal irradiation of the fluid, comprising:
via at least one fluid-moving element, moving a fluid through an inlet of a housing;
within the housing, slowing speed of a fluid flow of the fluid in at least one fluid flow expansion region;
via at least one fluid-flow blocking element, located within at least one of a plurality of chamber regions within the housing, producing a major region of turbulent fluid-flow in at least one of the chamber regions; and
via at least one radiation source that provides ultraviolet radiation, irradiating the fluid in at least one of the chamber regions thereby at least partially purifying the fluid.

Aptly the method further comprises providing at least one minor region of turbulent fluid flow within a respective chamber region of the housing via at least one shallow fluid flow blocking element in said a respective chamber region.

Aptly the method further comprises providing ultraviolet radiation via at least one ultraviolet light emitting diode (LED) in the housing or connected to the housing via a light guide.

Aptly the method further comprises diffusing radiation emitted from the LED via a lens element proximate to an emittance surface of the LED thereby flooding at least a portion of at least one chamber with UV radiation.

Aptly the method further comprises via a filter element filtering out UV radiation of a wavelength below 260 nm thereby only providing UV radiation in the housing with a wavelength of 260 nm or greater.

Aptly the fluid comprises air or oxygen.

Aptly the method further comprises moving the fluid through the housing via at least one fan or blower or pump.

Aptly the method further comprises narrowing a cross section of a fluid flow path through the housing in an anti-chamber of the housing prior to fluid flow from the anti-chamber to a first of the chamber regions and/or broadening a cross section of the flow path in an exit chamber of the housing subsequent to fluid flow from a final one of the chamber regions to the exit chamber.

Aptly the method further comprising continually recirculating fluid through the inlet and out of the outlet via the chamber regions thereby constantly purifying fluid in a vehicle that includes the housing.

Certain embodiments of the present invention thus provide a method and apparatus for at least partially purifying a fluid via germicidal radiation of the fluid.

According to certain embodiments of the present invention one or more ultraviolet LEDs can be utilised to provide UV germicidal irradiation (UVGI) to inactivate airborne pathogens and microorganisms like mould, bacteria, yeast and/or viruses from air and/or oxygen. Aptly short-wave ultraviolet light (UV-C light) is utilised.

Certain embodiments of the present invention utilise UV LED technology to reduce power consumption, improve efficiency, improve service cycles and reduce a physical size of a unit used to at least partially purify a fluid. This helps make the unit convenient for fit into travel interiors of vehicles such as aircraft or motorcars.

Certain embodiments of the present invention include a housing which combines reverse venturi effect to slow a velocity of airflow and one or more baffles that create turbulent airflow in a chamber within the housing to help maximise a time that fluid, such as air, is exposed within the housing to UV-C radiation. As a result any airborne pathogens and microorganisms are inactivated.

According to certain embodiments of the present invention multiple housings for partially purifying a fluid via germicidal irradiation can be fitted throughout a vehicle. For example on the back of each or every other seat in an aircraft. A constant recycling of air through multiple units helps achieve a very high degree of purification of fluid in the entire vehicle.

Certain embodiments of the present invention provide a controlled exposure using a combination of UV-C LEDs coupled with a venturi chamber and baffle design and also use materials for manufacturing a unit that include a high reflectivity to UV-C wavelengths. This helps ensure that one or more chambers in a housing slow the fluid to ensure a long exposure time at a relatively high dosage level. For example dosage levels of around 8000 mw·s/cm² can be achieved.

Certain embodiments of the present invention provide a unit which is scalable and thus can be sized to suit any application.

Certain embodiments of the present invention provide a dedicated personal air purification system for travel.

Certain embodiments of the present invention will now be described hereinafter, by way of example only, with reference to the accompanying drawings in which:

In the drawings like reference numerals refer to like parts.

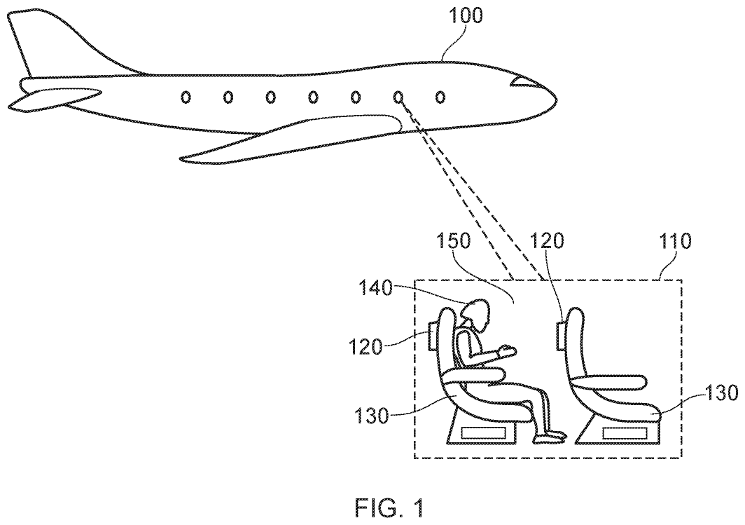

FIG. 1 illustrates a vehicle including an air purification system;

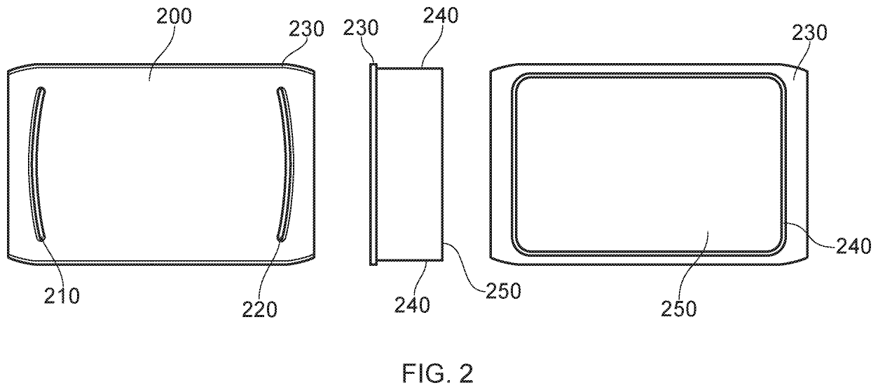

FIG. 2 illustrates a housing;

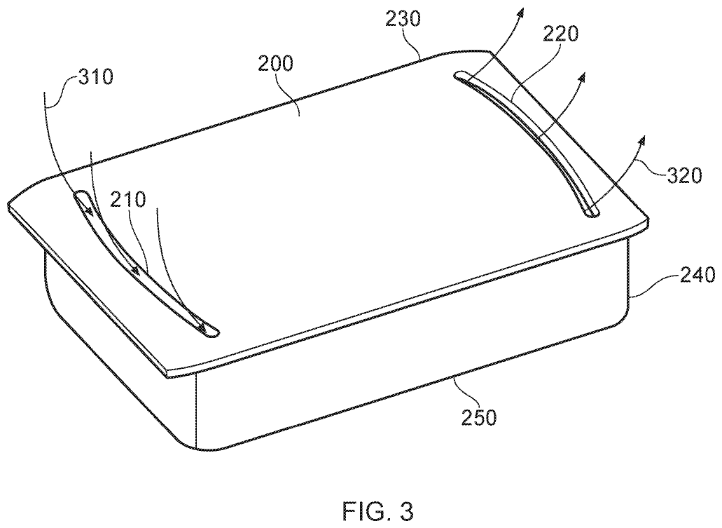

FIG. 3 illustrates an isometric view of a housing showing example inlet and outlet fluid flow paths;

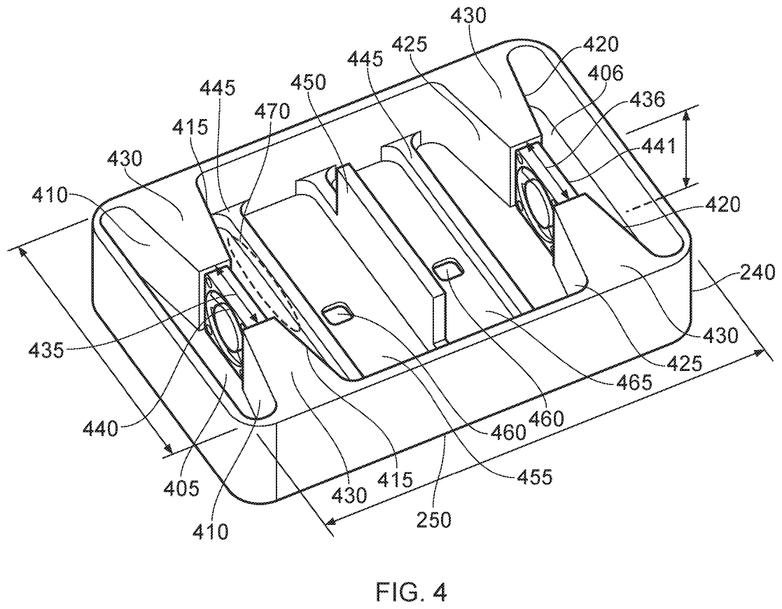

FIG. 4 illustrates an isometric view of an inside of a housing without a cover;

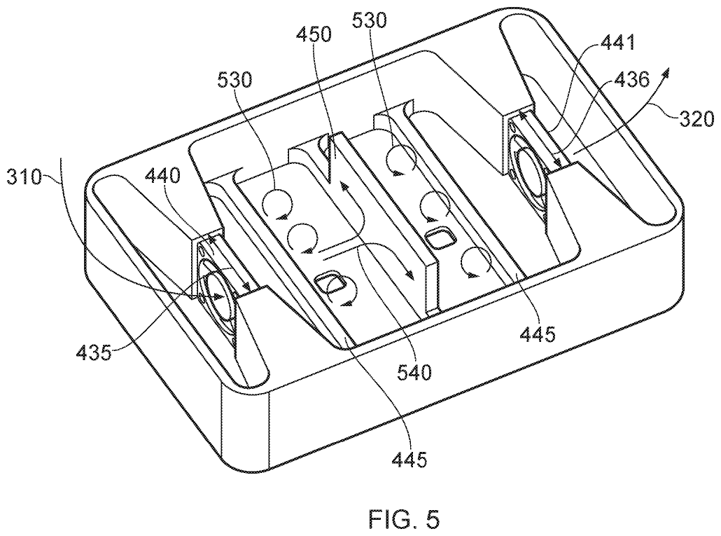

FIG. 5 illustrates an isometric view of an inside of a housing without a cover and showing example fluid flow paths;

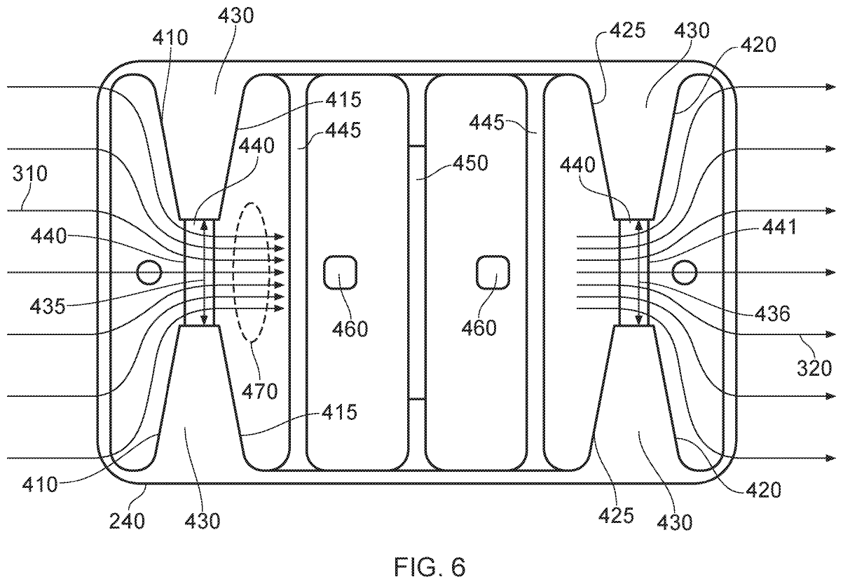

FIG. 6 illustrates a plan view of an inside of a housing without a cover and showing example fluid flow paths;

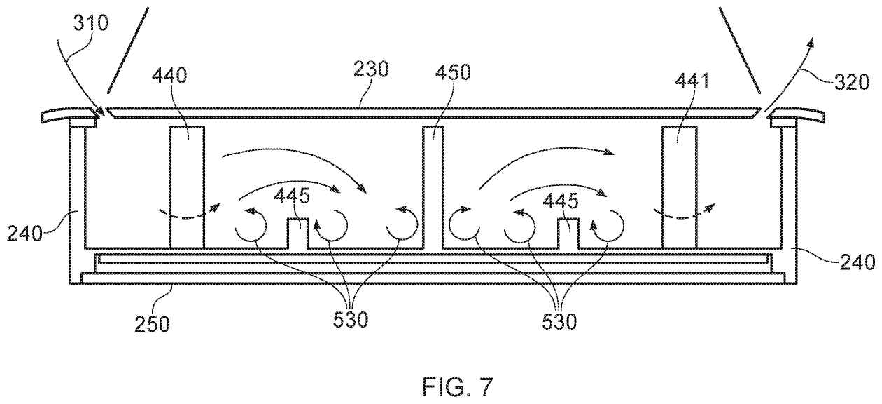

FIG. 7 illustrates a side view of a housing showing example fluid flow paths; and

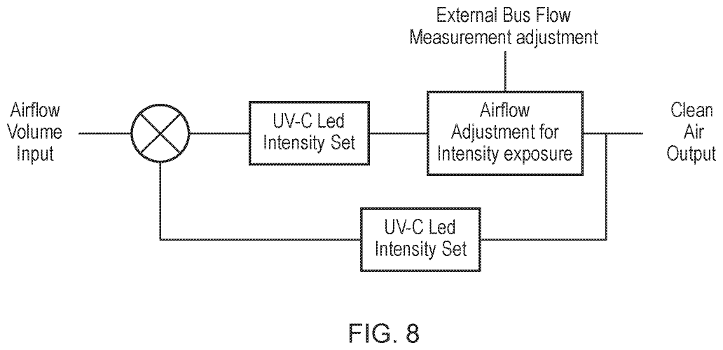

FIG. 8 illustrates a control diagram.

In the drawings like reference numerals refer to like parts.

FIG. 1 illustrates an example environment in which certain embodiments of the present invention may be utilised. An aeroplane 100 is an example of a vehicle and includes a section of seating 110 comprising seats 130. A germicidal air purifier 120 is attached to the back of each of the seats 130. The germicidal air purifier 120 takes in a portion of the surrounding air 150 via an inlet. While inside the air purifier 120, the air is irradiated to kill at least a subset of the microorganisms suspended in the air before expelling the at least partially purified air back into the surrounding air. A passenger 140 sits proximate to the germicidal air purifier 120.

It will be understood that certain embodiments of the present invention may be utilised in alternative environments. For example, the aeroplane 100 may instead be an alternative aircraft, such as a helicopter; a motor vehicle, such as a car/automobile or coach; a watercraft, such as a yacht or cruise ship; a train carriage; a hotel room; an office space; or a hospital ward. Similarly, the section of seating may instead be a bed, cabin, or suite within the aeroplane 100 or any of the aforementioned alternatives. The passenger 140 may alternatively be considered a user.

FIG. 2 shows a front provided by a cover, side, and rear view of a housing 200 of a germicidal air purifier 120. Surrounding air may be drawn into the housing 200 via fluid inlet 210 and may be expelled from the housing via fluid outlet 220. A cover 230 encloses the housing 200 which also comprises a side wall 240 and a base 250.

FIG. 3 show an isometric view of the housing 200. An example flow path of external air 310 entering the housing 200 via fluid inlet 210 is illustrated. The inlet shown is a through slit that is arcuate. Also illustrated is an example flow path of air that has been at least partially purified 320, which returns to the surrounding air via fluid outlet 220.

FIG. 4 shows an isometric view of the housing 200 excluding the cover 230. After passing through a fluid inlet 210, air enters an antechamber 405. The inclined wall portions 410 of the antechamber 405 taper from the side wall 240. The dividing wall portion 430 proximate to the antechamber 405 separate the antechamber 405 from the first of a plurality of consecutive chamber regions 455. These dividing walls 430 also provide a fluid flow aperture 435. Within the fluid flow aperture 435, there is a fan 440 for moving external air into the antechamber 405 via the fluid inlet 210 and toward a fluid outlet 220. Proximate to the fluid flow aperture 435, there is a first of a plurality of chamber regions 455.

Within the first chamber region 455, there is a fluid-flow expansion region 470 as well as further inclined walls 415 that taper from the side wall 240. The fluid-flow expansion region 470 is an example region wherein the fluid flow of the air may undergo a reverse Venturi effect. A baffle provides a full height fluid-flow blocking element 450 that separates the first chamber region 455 from a final chamber region 465 and at least partially blocks a fluid-flow between any two adjacent chamber regions, producing a major region of turbulent air in at least one of the two adjacent chamber region. The full height fluid-flow blocking element extends between 75% and 100% of a depth of a space between the base 250 and the cover 200 and has a length that is between 50% and 95% of a width between the side wall 240 on opposite sides of the housing 200. A bar provides a shallow fluid-flow blocking element 445 that is located within the first chamber region 455. A shallow fluid-flow blocking element 445 is also located within the final chamber region 465. The shallow fluid-flow blocking element 445 at least partially blocks the fluid-flow within the respective chamber regions and produces a minor region of turbulent fluid-flow within the respective chamber regions. The shallow fluid-flow blocking element 445 has a depth between 2% and 30% of a depth between the base 250 of the housing 200 and the cover 200. The shallow fluid-flow blocking element 445 may comprise a plurality of projections, such as boss-like, cone-like, pin-like, or dome-like protrusion. Within the final chamber region 465, there are inclined walls 425 that taper from the side wall 240.

The dividing wall portions 430 effectively provide a dividing wall proximate to the final chamber region 465 separate the final chamber region 465 from an exit chamber 406. These dividing walls 430 also provide a fluid flow exit aperture 436. Proximate to the exit chamber 406, the inclined walls 420 taper from the side wall 240. Within the fluid flow aperture 436, there is a fan 441 for moving air in the housing 200 out of the housing via fluid outlet 220.

The reverse Venturi effect and minor and major turbulent flow regions increase the amount of time taken for air to travel from the fluid-flow inlet 210 to the fluid-flow outlet 220 through the housing 200. Thus, a greater portion of the air flowing through the housing will be exposed to a sufficient dose of ultraviolent radiation to kill microorganisms suspended in the air flowing through the housing.

Two LEDs or two arrays of LEDs 460 are shown in the housing. These are each in a respective chamber region. They are provided by power and control signals via a remote or local controller.

FIG. 5 shows a fluid-flow path 310 of air being moving through the fluid-flow aperture 435 by fan 440. Minor regions of turbulent fluid-flow 530 produced by the shallow fluid-blocking element 445 are shown as is the major region of turbulent fluid-flow 540 produced by the full-height fluid-blocking element 445. An exit fluid-flow path 320 of air is also shown.

FIG. 6 illustrates another plan view of a housing for at least partially purifying a fluid via germicidal irradiation of the fluid. FIG. 6 illustrates how an inlet flow of fluid (shown on the left hand side of FIG. 6) flows through an inlet slit in a cover member of the housing into an antechamber. The antechamber is defined by an edge side wall and dividing wall portions and opposed surfaces of the dividing wall portions form a narrowing section to the antechamber. An aperture between the dividing wall portions includes a fan 440 which helps draw air in through the inlet aperture. The aperture has a cross section which is narrower than a cross section in the first chamber region 455. As a result airflow through the aperture is slowed as it enters the first chamber region by a reverse venturi effect this makes air dwell longer in the first chamber than it otherwise would without the narrowing region. Air flows through this first chamber region which is flooded by ultraviolet radiation from a first LED 460 which includes a lens to create diffuse light. The housing or surfaces in the housing can be made of a highly reflective material (in the UV range) to assist the flooding effect. The pathway through the housing straight between the inlet and the outlet is blocked by a central blocking element 450. This is generally centrally located along an axis associated with the housing so that airflow must be redirected around the sides of the blocking element (in FIG. 6 this is a baffle-like wall). Again this creates major turbulent flow helping to slow a velocity of airflow maximising an opportunity for pathogens and microorganisms and the like to be inactivated by the ultraviolet light. Fluid flow continues through a second chamber region 465 which includes a further LED flooding that chamber region. It will be appreciated by those skilled in the art that multiple cascaded chamber regions could of course be utilised. A further fluid moving element in the form of a fan is in an exit aperture between opposed wall portions that provide a dividing wall separating the final chamber region from an exit chamber. Fluid flows from the exit region and out of the housing via the arcuate opening in the cover layer.

FIG. 7 illustrates minor and major turbulent flow in the central volume of the housing.

FIG. 8 illustrates control of airflow and helps illustrate how airflow can be adjusted for intensity exposure so as to achieve a desired outcome.

UV-C LEDs

‘Doped’ lens for filtering unwanted high frequency UV radiation.

This is what inactivates the pathogens, The UV-C LED is factory tuned to the required wavelength by the LED manufacturer, The UV-C LED allows for an efficient chamber design that then makes it possible to install within the designed environments. There may be one or more UV-C LEDs depending on the chamber dimensions which is a function of the volume of air that has to be cleansed through the chamber. UV-C LEDs are used to allow accurate intensity control.

The choice of LED is based on the LENS having a wide radiance angle so that the UV light is diffused over the full area of the chamber.

Venturi Chamber

The Venturi chamber slows a volume of air of at least 133 cm\hat{ }3/second, such that when exposed to the UV-C LEDS, provide 8000μW·s/cm2 of UV exposure.

Piping

Piping in locations will provide air inlet/air outlet extension.

Baffles

Baffles within the Venturi chamber aid the exposure of air to the required 8000 μW·s/cm2 by causing Turbulence within the chamber and recirculating the air over the UV-C LEDs.

Pump(s)

The FAN(s) pump a controlled measurable consistent volume of air from the air inlet, through the Venturi chamber that incorporates the baffles out to the air inlet. Size of fan and numbers of fans depend on the chamber size, which is dependent on the final installed location.

Internal Hardware/Microelectronics, CANbus Interface, and Other Circuitry

A control bus such as CANbus or other bus interface provides remote control of the UV-C air purification system.

Control circuitry controls intensity of the UV-C LED vs airflow (FAN control) in the chamber, with airflow measurement being the control factor for setting the UV-C intensity and airflow volume.

The controller is located within the product housing, with the only external interfaces being power and control input, Control would be by a BUS system and provides control of FAN speed, which controls the volume of air being moved through the chamber. The Intensity of the UV-C Led source is controlled by the volume of air being sensed in the chamber. See the control diagram show in FIG. 8.

Certain embodiments of the present invention thus provide a compact UV air purification unit which utilises an internal luminated UV-C chamber that slows air entering the chamber to be less than 8 litres per minute (approximately the breathing rate of humans). This allows time for the UV-C at a required intensity to actively remove airborne pathogens and microorganisms thereafter returning clean air into a localised environment such as an aircraft cabin. UV-C can be utilised as an antimicrobial treatment and helps kill viruses, yeasts and moulds in the air. Aptly, short wavelengths of about 254 nm can be utilised. Aptly, short wavelengths of about 254 nm and upwards can be utilised. Aptly, short wavelengths of about 254 nm and greater can be utilised. At this wavelength ultraviolet light may be considered as germicidal and works by penetrating thin-wall germs like viruses and bacteria and fatally altering there genetic structure. A backlight can be included in a single colour or RGBWW and this can optionally be controlled via a CANbus or other controller interface if desired.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. Apparatus for at least partially purifying a fluid via germicidal irradiation of the fluid, comprising:
at least one fluid-moving element, located in a housing, for moving a fluid through at least one fluid inlet in the housing towards at least one fluid outlet in the housing, wherein the housing comprises a dividing wall that includes at least one fluid-flow aperture in a fluid-flow pathway between the inlet and the outlet, at least one fluid-flow expansion region, and a plurality of chamber regions;
at least one radiation source that provides ultraviolet radiation for irradiating the fluid in at least one of the chamber regions; and
at least one full height fluid-flow blocking element, each located between two adjacent chamber regions, for at least partially blocking a fluid-flow of fluid flowing between the two adjacent chamber regions and producing a major region of turbulent fluid-flow in at least one of the two adjacent chamber regions.

2. The apparatus as claimed in claim 1, further comprising:
at least one shallow fluid-flow blocking element, each located within a respective one of the chamber regions, for at least partially blocking a fluid-flow of fluid flowing within a respective chamber region and producing a minor region of turbulent fluid-flow within a respective chamber region.

3. The apparatus as claimed in any preceding claim, wherein:
the first fluid-moving element is located proximate to the fluid inlet or the fluid outlet and a further fluid-moving element is located proximate to a remainder of the fluid inlet or the fluid outlet for moving the fluid through the fluid outlet.

4. The apparatus as claimed in any preceding claim, wherein at least one radiation source comprises at least one radiation emitting element that emits ultraviolet radiation.

5. The apparatus as claimed in claim 4, wherein a light guide is disposed proximate to at least one radiation emitting element for diffusing emitted ultraviolet radiation.

6. The apparatus as claimed in any preceding claim, wherein at least one radiation source comprises:
a radiation filter that filters out and effectively removes ultraviolet radiation of a wavelength below 260 nanometres from the provided ultraviolet radiation.

7. The apparatus as claimed in any preceding claim, further comprising:
a controller unit, comprising a controller interface, connected to the at least one fluid-moving element and the at least one radiation providing device, for providing respective control signals to each fluid-moving element and radiation providing device.

8. The apparatus as claimed in any preceding claim, further comprising:
a power unit, comprising a power connection interface, connected to the at least one fluid-moving element and the at least one radiation providing device, for providing respective power signals to each fluid-moving element and radiation providing device.

9. The apparatus as claimed in claim 8, wherein:
the controller unit comprises the power unit.

10. The apparatus as claimed in claim 8 or claim 9, wherein:
the respective power signals comprise the respective control signals.

11. The apparatus as claimed in any one of claims 7 to 10, wherein:
the controller unit can include a Controlled Area Network (CAN) bus unit.

12. The apparatus as claimed in any preceding claim, further comprising:
the housing comprises a base member and an upstanding sidewall member extending around a perimeter of the base member, and a cover member that covers the housing.

13. The apparatus as claimed in claim 12 wherein each fluid inlet and each fluid outlet comprises a through hole in the cover member.

14. The apparatus as claimed in claim 13, further comprising:
the fluid inlet and/or the fluid outlet comprises a slit or a row of discrete holes in the cover member and optionally the row of discrete holes or the slit is arcuate.

15. The apparatus as claimed in any one of claims 12 to 14, further comprising:
each full height fluid-flow blocking element comprises a wall member upstanding from the base member or extending from the cover member and that extends between 75% and 100% of a depth of a space between an inner surface of the base member and an inner surface of the cover member.

16. The apparatus as claimed in claim 15, further comprising:
each full height fluid-flow blocking element has a length that is between 90% and 50% of a width between the side wall on opposed sides of the housing.

17. The apparatus as claimed in claim 2, further comprising:
each shallow fluid-flow blocking element comprises a bar member that extends across a whole or a portion of a width of the housing between a side wall on opposed sides of the housing and each bar member is proximate to a base member or to a cover member of the housing and has a depth between 2% and 30% of a depth of a space between an inner surface of a base member of the housing and an inner surface of a cover member of the housing.

18. The apparatus as claimed in claim 2, further comprising:
each shallow fluid-flow blocking element comprises one of a plurality of projections that extend from an inner surface of a cover member of the housing and/or of a base member of the housing.

19. The apparatus as claimed in claim 18, further comprising:
each projection is a boss-like or cone-like or pin-like or dome-like element.

20. The apparatus as claimed in any preceding claim wherein the apparatus includes at least one full height fluid-flow blocking element and/or at least one shallow fluid-flow blocking element and each blocking element comprises at least one vortex shedding site.

21. The apparatus as claimed in any preceding claim, further comprising:
the housing comprises an antechamber between the fluid inlet and a first of the chamber regions and the housing includes mutually inclined walls that face an interior of the antechamber proximate to the fluid-flow aperture thereby providing a narrowing of the antechamber proximate to the fluid-flow aperture wherein the narrowing antechamber is narrower proximate to the fluid-flow aperture than distal to the fluid-flow aperture.

22. The apparatus as claimed in any preceding claim, further comprising:
the housing comprises an exit chamber between a final chamber region of the chamber regions and the fluid outlet and the housing includes mutually inclined walls that face an interior of the exit chamber proximate to an exit aperture in a dividing wall between the final chamber region and the exit chamber thereby providing a broadening out of the exit chamber proximate to the exit aperture, the broadening out of the exit chamber being narrower proximate to the exit aperture and extending in a flared out arrangement towards a central region of the exit chamber.

23. A vehicle comprising the apparatus as claimed in any preceding claim.

24. A method for at least partially purifying a fluid via germicidal irradiation of the fluid, comprising:
via at least one fluid-moving element, moving a fluid through an inlet of a housing;
within the housing, slowing speed of a fluid flow of the fluid in at least one fluid flow expansion region;
via at least one fluid-flow blocking element, located within at least one of a plurality of chamber regions within the housing, producing a major region of turbulent fluid-flow in at least one of the chamber regions; and
via at least one radiation source that provides ultraviolet radiation, irradiating the fluid in at least one of the chamber regions thereby at least partially purifying the fluid.

25. The method as claimed in claim 24, further comprising:
providing at least one minor region of turbulent fluid flow within a respective chamber region of the housing via at least one shallow fluid flow blocking element in said a respective chamber region.

26. The method as claimed in claim 24 or claim 25, further comprising:
providing ultraviolet radiation via at least one ultraviolet light emitting diode (LED) in the housing or connected to the housing via a light guide.

27. The method as claimed in claim 26, further comprising:
diffusing radiation emitted from the LED via a lens element proximate to an emittance surface of the LED thereby flooding at least a portion of at least one chamber with UV radiation.

28. The method as claimed in any one of claims 23 to 27, further comprising:
via a filter element filtering out UV radiation of a wavelength below 260 nm thereby only providing UV radiation in the housing with a wavelength of 260 nm or greater.

29. The method as claims in any one of claims 24 to 28 wherein the fluid comprises air or oxygen.

30. The method as claimed in any one of claims 24 to 29, further comprising:
moving the fluid through the housing via at least one fan or blower or pump.

31. The method as claimed in any one of claims 24 to 30, further comprising:
narrowing a cross section of a fluid flow path through the housing in an anti-chamber of the housing prior to fluid flow from the anti-chamber to a first of the chamber regions and/or broadening a cross section of the flow path in an exit chamber of the housing subsequent to fluid flow from a final one of the chamber regions to the exit chamber.

32. The method as claimed in any one of claims 24 to 31, further comprising continually recirculating fluid through the inlet and out of the outlet via the chamber regions thereby constantly purifying fluid in a vehicle that includes the housing.
